(19) United States
(12) Patent Application Publication
(10) Pub. No.: US 2024/0207217 A1
(43) Pub. Date: Jun. 27, 2024

(54) IMPROVED TREATMENT METHODS FOR CABAZITAXEL

(71) Applicant: ORBICULAR PHARMACEUTICAL TECHNOLOGIES PRIVATE LIMITED, Hyderabad (IN)

(72) Inventors: Bhaveshkumar VALLABHBHAI PATEL, Hyderabad (IN); Raghu KANNEKANTI,, Hyderabad (IN)

(21) Appl. No.: 18/569,581

(22) PCT Filed: Oct. 30, 2021
(86) PCT No.: PCT/IB2021/060071

§ 371 Date: Dec. 12, 2023

(30) Foreign Application Priority Data
Oct. 30, 2020 (IN) 202041032697

(51) Int. Cl. A61K 31/337

(52) U.S. Cl. CPC A61K 31/337

(57) ABSTRACT

An improved method of treatment with cabazitaxel composition that is substantially free of premedication prior to the administration of the cabazitaxel composition to the patient.

### FIELD OF THE INVENTION

The present invention relates to method of treating a patient in need of cabazitaxel comprising parenterally administering to said patient an aqueous composition of cabazitaxel treatment, wherein the treatment is substantially free of premedication prior to the administration of the cabazitaxel composition.

### BACKGROUND OF THE INVENTION

Antineoplastic agents inhibit and combat the development of neoplasms, which are abnormal masses of tissue resulting from irregular proliferation of cells. Such antineoplastic agents include taxane compounds that play an important role in the treatment of various solid tumors. These chemotherapeutic taxane derivatives include paclitaxel, docetaxel and cabazitaxel which are well known and established drugs in the treatment of malignant tumors. Since the initial approval of Paclitaxel (Taxol®; Bristol-Myers Squibb, New York City, NY, USA) in 1992, the first generation taxanes, paclitaxel and docetaxale (Taxotere®; Sanofi, Paris, France) have altered the treatment paradigm for a wide variety of solid tumors, including breast, lung, prostate, gastric and ovarian cancers. Docetaxel was the first cytotoxic therapy associated with a survival benefit in castration-resistant prostate cancer but the majority of patients were found to eventually acquire resistance to docetaxel therapy.

Cabazitaxel is a novel second-generation taxane derivative prepared by semi-synthesis with a precursor extracted from yew needles (10-deacetylbaccatin Ill). Cabazitaxel binds to free tubulin and promotes the assembly of microtubules, which reduces the availability of tubulin for, and thereby prevents, cell division. Simultaneously, cabazitaxel inhibits microtubule disassembly, causing apoptosis. Cabazitaxel (Jevtana®, Sanofi) was developed by Sanofi-Aventis and approved by U.S. FDA on Jun. 17, 2010, in combination with prednisone for the treatment of patients with metastatic hormone-refractory prostate cancer who have previously received docetaxel-based therapy.

Taxol is the brand name for Paclitaxel and is available as a clear, colourless to slightly yellow viscous injection solution that is supplied as a nonaqueous solution intended for dilution with a suitable parenteral fluid prior to intravenous infusion. TAXOL is available in 30 mg (5 mL), 100 mg (16.7 mL), and 300 mg (50 mL) multidose vials. Each mL of sterile nonpyrogenic solution contains 6 mg paclitaxel, 527 mg of purified Cremophor® EL* (polyoxyethylated castor oil) and 49.7% (v/v) dehydrated alcohol, USP. Taxotere is the brand name for docetaxel injection that is available as a sterile, non-pyrogenic, pale yellow to brownish yellow solution at 20 mg/ml concentration.

Each mL contains 20 mg docetaxel (anhydrous) in 0.54 grams polysorbate 80- and 0.395-grams dehydrated alcohol (50% v/v) solution, with citric acid for pH adjustment.

Jevtana Kit (cabazitaxel) injection 60 mg/1.5 ml is a sterile, non-pyrogenic, clear yellow to brownish yellow viscous solution and is available in single-dose vials containing 60 mg cabazitaxel (anhydrous and solvent free) and 1.56 g polysorbate 80.

Taxanes are an important cause of hypersensitivity reactions (HSRs) in cancer patients. Consequently, to avoid the risk of such side effects, a prophylactic treatment with antihistamines and corticosteroids is necessary prior to the treatment with taxanes. The prophylactic medication may reduce the severity of the hypersensitivity reaction, but cannot completely eliminate it. For instance, paclitaxel (taxol) causes immediate hypersensitivity reactions (HSRs) in around 10% of patients despite premedication with antihistamines and a corticosteroid.

Hypersensitivity reactions to paclitaxel have been reported since the phase I trial. But it is still inconclusive whether cremophor or paclitaxel itself causes hypersensitivity reactions. However, it is widely recognized that hypersensitivity reactions are caused by cremophor (as detailed below) rather than paclitaxel itself.

Paclitaxel (compounded with Cremophor EL) has been shown to cause a marked complement activation in vitro in 4/10 healthy subjects and 5/10 cancer patients at a concentration of 100 μg/ml, which translates into a Cremophor EL concentration of 8.8 mg/ml1. In comparison, the maximal serum concentration of Cremophor EL reached with a 3-hour paclitaxel infusion at a dose of 175 mg/m² averages 3 mg/mL. Furthermore, Cremophor EL alone was found to trigger complement activation to the same extent that paclitaxel/Cremophor EL did at equivalent concentrations suggesting that Cremophor EL is solely responsible for this effect. Similarly, docetaxel (compounded with polysorbate 80) and polysorbate 80 alone have been shown to cause complement activation in vitro.

These findings support the role of these emulsifying agents in triggering complement activation invitro and it is hypothesized that through production of anaphylatoxins they could trigger immediate HSRs in vivo, thereby explaining why most patients experience immediate HSRs on first exposure to taxanes if no premedication i.e antihistamines and corticosteroids are used.

The taxane compounds, paclitaxel, docetaxel and cabazitaxel are poorly soluble molecules in water. Therefore, to allow intravenous administration, each of the marketed products of taxane compounds as stated above include emulsifying agents in their formulation such as polyethoxylated castor oils (Cremophor EL) for paclitaxel and Poly-oxyethylene-sorbitan monooleate (Polysorbate 80) for docetaxel and cabazitaxel. U.S. Pat. Nos. 5,403,858 A, 5,698,582 A, 5,922,754 A, and Auspar disclose the use of polysorbates, polyoxyethylene glycol esters and polyethoxylated castor oils as suitable surfactants, respectively.

The presence of Cremophor in Taxol has been linked to severe hypersensitivity reactions. A patient receiving paclitaxel in the widely used dose of 175 mg/m² will also receive about 14 ml/m² of Cremophor. Cremophor EL has been associated with bronchospasm, hypotension and other manifestations of hypersensitivity. In phase 1 clinical trials of Taxol, severe hypersensitivity reactions were reported in as many as 18% patients. In phase II trials, a 24-hour schedule for intravenous administration was used along with oral or intravenous dexamethasone (6 and 12 hours pretreatment), diphenhydramine and an intravenous H2 blocker, usually cimetidine (30 mins pretreatment) that decreased the incidence of serious anaphylactic reactions to 3% or less (Arbuck et. al., Christian et. al, Clinical Development of Taxol; J, NCI: Monographs, Issue 15).

Koeller et. al. in “A Phase I Pharmacokinetic Study of Taxol given by a prolonged infusion without premedication”, recommended routine premedication in order to avoid the significant number of allergic reactions believed to be caused by the cremophor (polyethoxylated castor oil) vehicle used for taxol infusions.

Weiss R B, Donehower R C, Wiernik P H, et al. in “Hypersensitivity reactions from Taxol. J Clin Oncol 1990, 8, 1263-1268”, stated that severe hypersensitivity reactions in humans were associated with presence of cremophor in the formulation.

Samelis G et al., study titled Premedication one hour before the treatment with taxanes, in Journal Prevention of therapy-related side effects September 1997 discloses, allergic reactions are the most common and serious side effect of treatment with taxanes. The premeditation with histamine H1, H2 antagonists and dexamethasone is established and suggested to start 13 hours before taxane administration. Two hundred and one patients were reviewed. 169 were treated with Paclitaxel and 32 With Docetaxel. One thousand and one hundred ninety eight courses were administered. In 300 courses the premedication with dexamethasone and difenidramine, started 13 hours before the administration of Pacliaxel (or Docetaxel). In 898 courses the premedication was given one hour before Taxanes. No differences in allergic reactions were seen between the two different timing of premedication. These reactions were equally distributed in both ways of premedication. Premedication with anti-allergic drugs one hour before the administration of taxanes has no consequences and can be considered safe.

J S Kloover, et al., Br J Cancer 2004, titled Fatal outcome of a hypersensitivity reaction to paclitaxel: a critical review of premedication regimens discloses Hypersensitivity reactions (HSRs) to paclitaxel are frequently encountered in patients receiving this antitumour drug. Administration of histamine H1- and H2-receptor antagonists and corticosteroids has been shown to reduce significantly the risk of developing an HSR in patients receiving taxanes.

Ping Ma, et al., J Nanomed Technology, February 2013, discloses paclitaxel is one of the most effective chemotherapeutic drugs ever developed and is active against a broad range of cancers, such as lung, ovarian, and breast cancers. Due to its low water solubility, paclitaxel is formulated in a mixture of Cremophor EL and dehydrated ethanol (50:50, v/v) a combination known as Taxol. However, Taxol has some severe side effects related to Cremophor EL and ethanol. Therefore, there is an urgent need for the development of alternative Taxol formulations.

Common Chemotherapy Drug Can Trigger Fatal Allergic Reactions.” ScienceDaily. ScienceDaily, 9 Jun. 2009. A new study from the Research on Adverse Drug Events and Reports (RADAR) pharmacovigilance program at Northwestern University Feinberg School of Medicine identified 287 unique cases of hypersensitivity reactions submitted to the FDA's Adverse Event Report System between 1997 and 2007 with 109 (38 percent) deaths in patients who received Cremophor-based paclitaxel, a solvent-administered taxane chemotherapy.

Adverse event reports generally only represent from 1 to 10 percent of actual incidence, so the number of hypersensitivity reactions and deaths is likely significantly higher. The severe allergic reactions are believed to be caused by Cremophor, the chemical solvent—a derivative of castor oil—that is used to dissolve some insoluble drugs before they can be injected into the blood stream.

The current regimen of administration for Taxol, for this reason involves treatment of the patient with antihistamines, H2 blockers and steroids prior to the injection of the drug to reduce the allergic side effects of Cremophor.

Docetaxel is a taxane that inhibits cell replication by stabilizing the microtubule cytoskeleton. To solubilize this agent for clinical use, it has been formulated using polysorbate 80. An injection concentrate (20 mg/ml) comprises 20 mg of docetaxel in 1 ml at a 50/50 (V/V) ratio of polysorbate 80/dehydrated alcohol. Docetaxel has been associated with a number of infusion-site adverse events (ISAEs) and systemic reactions including hypersensitivity fluid retention, cutaneous reactions, gastrointestinal adverse events, and peripheral neuropathy. In clinical trials in patients with cancer, the incidence of ISAEs has been reported to be 3-4%.

The presence of polysorbate 80 in the IV formulation of docetaxel (Taxotere) has been implicated in hypersensitivity systemic reactions (HSRs) that were observed in the early clinical studies. In those studies, the incidence of HSRs ranged from 5% to 40%, with most events being grade 2 in severity on the four-point scale of the National Cancer Institute common toxicity criteria. Severe HSRs or, very rarely, fatal anaphylaxis has been reported in patients treated with docetaxel. Polysorbate 80 and the oleic acid released by its metabolism have been implicated in the HSRs seen with docetaxel therapy.

Docetaxel therapy mandates premedication with steroids such as dexamethasone, used in the dosage regimen, in order to suppress hypersensitivity reactions.

All patients should be premedicated with oral corticosteroids such as dexamethasone 16 mg per day (e.g., 8 mg twice daily) for 3 days starting 1 day prior to TAXOTERE administration in order to reduce the incidence and severity of fluid retention as well as the severity of hypersensitivity reactions.

For metastatic castration-resistant prostate cancer, given the concurrent use of prednisone, the recommended premedication regimen is oral dexamethasone 8 mg at 12 hours, 3 hours, and 1 hour before the TAXOTERE infusion.

Cabazitaxel is increasingly used in cancer therapy because of its superior pharmacological properties and low drug resistance (ZHOU et. al., Jin et. al “Anticancer Research, 2016, 36, 1649-1656).

The presence of Polysorbate 80 in Jevatana Kit has the potential to cause hypersensitivity reactions. Clinical manifestations of cabazitaxel-associated hypersensitivity reactions include hypotension, bronchospasm, rash, and erythema or very rarely fatal anaphylaxis have been reported in patients treated with cabazitaxel. These, manifestations usually develop within minutes of the infusion, especially during the first and second infusions, and are reversible, resolving after the infusion is withheld (Dr. Nightingale et. al. P&T, 2012 August, 37(8): 440-442, 445-448).

Even though, the amount of polysorbate 80 entering the body through Jevtana is comparatively less than docetaxel formulation (taxotere) with respect to dosage based on calculation of the body surface area, infusion of Jevtana is still associated with hypersensitivity reactions.

Hypersensitivity reactions may require immediate discontinuation of the cabazitaxel infusion and patients may stop cabazitaxel therapy, skip a dose, or continue further therapy at a reduced dose. In order to reduce the side effects of hypersensitivity induced by Polysorbate 80 in cabazitaxel formulation (Jevtana), premedication with corticosteroids, antihistamines and H2 antagonists in patients has been recommended prior to administration of each dose of cabazitaxel. Dexamethasone, recommended as a premedication for JEVATANA, is a corticosteroid that suppresses the immune response in patients which can be detrimental in cancer patients under chemotherapy whose immunity is already compromised due to destruction of healthy cells by chemotherapeutic treatment. Also, it has drawbacks as corticosteroids have several other side effects and are not able to be used in diabetic patients. Hence, such premedication with corticosteroids is not desired, especially in patients with cancer.

Polysorbate 80 is a synthetic nonionic surfactant used as an excipient in drug formulation. Lee S. Schwartzberg. Rudolph M. Navari in “Adv Ther (2018) 35:754-767; Safety of Polysorbate 80 in the Oncology Setting” disclosed that enzyme-linked immunosorbent assays have shown that polysorbate 80 could activate the complement system, a multiprotein immune mechanism. Activating the complement system may lead to phagocytosis, stimulation, and recruitment of white blood cells, or perforation of plasma membranes, possibly leading to immunologic side effects such as acute hypersensitivity and systemic immune reactions.

Considering the above stated drawbacks due to the presence of Cremophor EL and Polysorbate 80 in taxane formulations, extensive research has been carried out to develop alternative delivery forms. Currently, several strategies are in progress to develop Polysorbate 80- and Cremophor EL-free formulations of docetaxel, paclitaxel and cabazitaxel which are based on pharmaceutical (e.g. albumin nanoparticles, emulsions and liposomes), chemical (e.g. polyglutamates, analogues and prodrugs), or biological (e.g. oral drug administration) strategies (Ten Tije, et. al., Clinical Pharmacokinetics, 2003; 42(7):665-85).

U.S. Pat. No. 6,506,405 patent discloses a paclitaxel formulation for treatment of primary tumors in a subject which achieves high local concentration of said paclitaxel at the tumor site, said formulation being substantially free of cremophor and comprising paclitaxel in a dose in the range of about 30 mg/m² to about 1000 mg/m².

Nuhad K. Ibrahim, et al., Clin Cancer Res. 2002 May; 8(5):1038-44Phase I and Pharmacokinetic Study of ABI-007, a Cremophor-free, Protein-stabilized, Nanoparticle Formulation of Paclitaxel; discloses clinical trial was conducted to examine the pharmacokinetic properties and spectrum of toxic effects associated with ABI-007. Because ABI-007 is not formulated in a Cremophor-containing solvent, we anticipated that hypersensitivity reactions would be diminished or absent. Our results show that ABI-007 can indeed be administered safely as a short infusion without dexamethasone or antihistamine premedication. Thus, when considering the process of drug administration, ABI-007 appears to offer advantages in terms of safety (avoidance of hypersensitivity reactions), morbidity (avoidance of dexamethasone premedication), and patient convenience and comfort (less time spent in the treatment center). These advantages could ultimately translate into an overall decrease in cost of therapy.

US2012065255 A1 discloses a pharmaceutical formulation of cabazitaxel with solubilizer, wherein the solubilizer is selected from glycofurol, ethanol and tocopherol polyethylene glycol succinate (TPGS) wherein the formulation is free of polysorbates and polyethoxylated castor oil.

WO2016149162 A1 publication disclose liquid pharmaceutical composition comprising, a taxane complexed with a beta-cyclodextrin or beta-cyclodextrin derivative, a low molecular weight polyethylene glycol, an alcohol suitable for parenteral administration, and water.

WO2014122498 A1 is directed to a composition comprising cabazitaxel and sulfobutyl ether beta cyclodextrin, wherein the composition is free of ethanol or surfactants.

WO2018109731 A1 discloses a pharmaceutical composition comprising Cabazitaxel or its derivatives, cyclodextrins derivative, and optionally with other pharmaceutically acceptable excipients, wherein, a) the composition is free of surfactants and glycols, b) the composition is ready to dilute, ready to use, ready to infuse compositions, c) the composition is in the concentration of o about 0.1 mg/mL to about 7.0 mg/mL; the compositions are administered in low volumes, with increased rate of administration and reduced administration duration to the patient.

U.S. Pat. No. 9,744,246 B2 discloses macromolecule comprising a dendrimer comprising a core and at least one generation of building units, the outermost generation of building units having surface amino groups wherein the pharmaceutically active agent is cabazitaxel and is free of Polysorbate 80 or Cremophor EL.

WO2020014750 A1 discloses a therapeutic dendrimer comprising a core and building units, the dendrimer further comprising a plurality of first terminal groups (TI) each comprising a cabazitazel residue covalently attached to a diglycolyl linker group and hence do not require any surfactants.

US2019224332 A1 discloses sterile injectable composition comprising one or more drugs, 1,2-dimyristoyl-sn-glycero-3-phosphorylglycerol (DMPG), N-(carbonyl-methoxypolyethyleneglycol)-1,2-distearoyl-sn-glycero-3-phosphoethanolamine (mPEG2000-DSPE) and one or more stabilizers wherein one of the drugs is cabazitaxel. Such formulation is also devoid of surfactants such as Polysorbate 80 and Cremophor EL.

Several literature comprising cabazitaxel that are free of surfactants such as polyethoxylated sorbitan fatty ester like polyoxyethylene sorbitan monooleate (polysorbate 80) and Polyethoxylated castor oil (Cremophor EL) are disclosed, however these literatures are silent on the premedication therapy. Premedication therapy before Cabazitaxel or Taxane treatment is quintessential due to the complement activation followed by hypersensitivity reactions caused by the surfactants and/or cremophor. Hence, there remains a need for treatment of Cabazitaxel that is free of premedication prior to the administration of the Cabazitaxel to the patient. The inventors of the present invention have surprisingly and unexpectedly found a method of treating a patient in need of cabazitaxel treatment, substantially free of premedication to control complement activation, reduce the toxicity and hypersensitivity reactions associated with the surfactants.

### SUMMARY OF THE INVENTION

The objective of the present invention is to provide a method of treating a patient in need of cabazitaxel comprising parenterally administering to said patient an aqueous composition of cabazitaxel comprising, cabazitaxel or a pharmaceutically acceptable salt thereof wherein the treatment is substantially free of premedication, required to reduce the Hypersensitivity reactions associated with excipients like surfactants.

In one aspect of the present invention, the premedication therapy is free of antihistamine, prior to the administration of cabazitaxel composition.

In one aspect of the present invention, antihistamine includes dexchlorpheniramine, diphenhydramine, prochlorperazine, and the like.

In one aspect of the present invention, the premedication therapy is administered to reduce the hypersensitivity reactions associated with excipients like surfactants.

In one aspect of the present invention the method of treatment comprises administration of an aqueous composition of cabazitaxel that is free of surfactants to reduce the hypersensitivity reactions associated with surfactants, wherein surfactants are selected from polysorbate 80, polyethoxylated castor oil and/or combinations thereof.

In one aspect, method of treating a patient in need of cabazitaxel treatment, substantially free of premedication to control complement activation, reduce the toxicity and hypersensitivity reactions associated with the surfactants.

In another aspect of the present invention, it is a method for improving the cabazitaxel treatment to a patient in need of cabazitaxel comprising, parenterally administering to said patient an aqueous composition of cabazitaxel comprising, cabazitaxel or a pharmaceutically acceptable salt thereof wherein the treatment is substantially free of premedication, required to reduce the hypersensitivity reactions associated with excipients like surfactants.

In one aspect of the present invention, the method of treatment comprises administration of an aqueous composition of cabazitaxel wherein the premedication therapy is free of antihistamine, prior to the administration of cabazitaxel composition.

In one aspect of the present invention, the premedication therapy is free of antihistamine wherein antihistamine includes dexchlorpheniramine, diphenhydramine, prochlorperazine and the like.

In another aspect of the present invention, the premedication therapy is administered to reduce the hypersensitivity reactions associated with excipients like surfactants or Cremophor.

In one aspect of the present invention, the composition is free of surfactants to reduce the hypersensitivity reactions associated with surfactants, wherein surfactants are selected from polysorbate 80, polyethoxylated castor oil and/or combinations thereof.

In one aspect of the present invention the therapy is for treatment of Cabazitaxel sensitive diseases.

In one aspect of the present invention, the aqueous composition of cabazitaxel comprises other excipients, wherein the excipients include solvents and/or co-solvents, solubilizer, pH modifiers, buffers, preservatives and combinations thereof.

In another aspect of the present invention, the aqueous composition of cabazitaxel comprises a solvent selected from water for injection, alkanol, water for injection; solubilizer include cyclodextrin and its derivatives; buffers include ascorbic acid, boric acid, citric acid, tartaric acid, lactic acid, oxalic acid, formic acid and the like, preservatives include ascorbic acid, acetylcysteine, sulfurous acid salts (bisulfite, metabisulfite), monothioglyercol, phenol, metacresol, benzyl alcohol, parabens (methyl, propyl, butyl), benzalkonium chloride, chlorobutanol, thimerosal and combinations thereof.

### DETAILED DESCRIPTION OF FIGURES

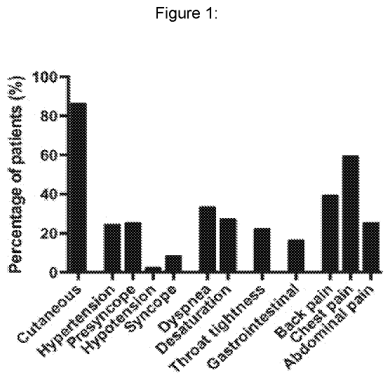

FIG. 1: Clinical features of immediate HSRs to taxanes. Created with data from J Allergy Clin Immunol. 2008; 122:574-80 and Gynecol Oncol. 2005; 99:393-9

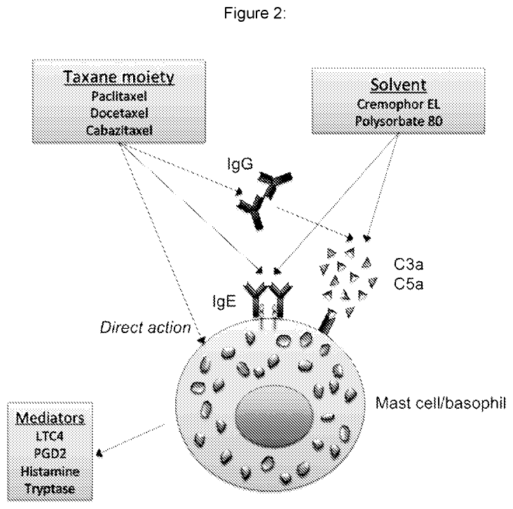

FIG. 2: Mechanisms of immediate HSRs to taxanes. The taxane moiety may cause mast cell and/or basophil activation through three different mechanisms: (1) a direct but undefined action on basophils but not mast cells; (2) an IgE-mediated mechanism, or (3) an IgG-mediated mechanism that causes complement activation through immune complex formation leading to the production of anaphylatoxins (C3a and C5a) that can activate basophils and mast cells through their surface receptors. Solvents may cause mast cell and/or basophil activation through two different mechanisms: (1) an IgE-mediated mechanism or (2) direct complement activation that leads to the production of anaphylatoxins. Following activation, mast cells and basophils release a variety of mediators that can be measured (serum tryptase or 24-h urinary methylhistamine), and the actions of which can be pharmaceutically blocked (histamine antihistamines; LTC4 montelukast; PGD2 aspirin). LTC4 leukotriene C4. PGD2 prostaglandin D2.

### DETAILED DESCRIPTION OF THE INVENTION

The subject invention is related to a method of treating a patient in need thereof by parenteral administration of an aqueous composition of cabazitaxel wherein the treatment is substantially free of premedication prior to the administration of the composition of cabazitaxel to the patient.

The term “composition” is intended to encompass a combination including active ingredients and pharmaceutically acceptable excipients, as well as any product which results, directly or indirectly, from combination, complexation, or aggregation of any two or more of the ingredients, or from dissociation of one or more of the ingredients, or from other types of reactions or interactions involving one or more of the ingredients.

The term “formulation” or “dosage form” or “composition” refers to finished pharmaceutical products that are suitable for administration, including, but not limited to, injections, parenteral, and the like. The term composition relates to aqueous compositions and or its related compositions as known in the art.

As used herein, the term “substantially free of premedication” or “free of premedication” is referred to the prior medication therapy administered before Cabazitaxel treatment. General premedication includes administration of an antihistamine, a corticosteroid and H2 antagonist before Cabazitaxel treatment. As used herein, the premedication is substantially free of antihistamine before Cabazitaxel treatment; the premedication is free of antihistamine before Cabazitaxel treatment.

As used herein, the term “Hypersensitivity reactions” or “Hypersensitivity Systemic Reactions” (HSRs) refer to reactions associated on complement activation with the administration on surfactants like polyethoxylated sorbitan fatty ester like polyoxyethylene sorbitan monooleate (polysorbate 80) and/or Polyethoxylated castor oil (Cremophor EL). Clinical manifestations of hypersensitivity reactions include complement activation. The other adverse effects include hypotension, bronchospasm, rash, and erythema.

As used herein, the term “Surfactants” refers to surface active agents, substance such as a detergent that, when added to a liquid, reduces its surface tension, thereby increasing its spreading and wetting properties. Surfactants for the purpose of this invention includes polyethoxylated sorbitan fatty esters like polyoxyethylene sorbitan monooleate (polysorbate 80) and/or Polyethoxylated castor oil (Cremophor EL) and the like.

The term “active ingredient” or “drug” refers to a substance that has a physiological effect when ingested or otherwise introduced into the body, in-particular or a chemical substance used in the treatment, cure, prevention, or diagnosis of disease or used to otherwise enhance physical or mental wellbeing.

The term “excipient” or “pharmaceutically acceptable excipient” or “adjuvant” means a component of a pharmaceutical product that is not a pharmacologically active ingredient, such as diluent, bulking agent, carrier, acidifying agent, pH modifier, chelating agent, solvent, co-solvent, tonicity modifier, antioxidant, preservative and the like added to a drug to increase or aid its effect. The excipients or adjuvants that are useful in preparing pharmaceutical compositions are generally safe, non-toxic, and neither biologically nor otherwise undesirable and are acceptable for veterinary use as well as human pharmaceutical use. The term includes one or more excipients or adjuvants.

The term “optional” or “optionally” means that the subsequently described element, component or circumstance may or may not be present, so that the description includes instances where the element, component, or circumstance is included and instances where it is not.

The formulation of the present invention is particularly suitable for parenteral administration. Formulations suitable for parenteral dosage forms such as injectable like intravenous, intramuscular or subcutaneous, implants and the like. Other parenteral ingredients used in the formulation are generally those commonly used and recognized by persons skilled in the art of parenteral formulations.

“Pharmaceutically-acceptable salts” refer to derivatives of the disclosed compounds wherein the parent compound is modified by making acid or base salts, solvate, hydrate, esters and the like thereof. Pharmaceutically acceptable salt forms of compounds provided herein are synthesized from the parent compound which contains a basic or acidic moiety by conventional chemical methods. Pharmaceutically acceptable salts are those forms of compounds, suitable for use in contact with the tissues of human beings and animals without excessive toxicity, irritation, allergic response, or other problem or complication, commensurate with a reasonable benefit/risk ratio.

It must be noted that, as used in this specification and the appended claims, the singular forms “a,” “an” and “the” include plural references unless the context clearly dictates otherwise. Thus, for example, reference to “an excipient” includes a single excipient as well as two or more different excipients, and the like.

Any recitation of ranges of values set forth below is merely intended to serve as a shorthand method of referring individually to each separate value falling within the range, unless otherwise indicated herein, and each separate value is incorporated into the specification as if it were individually recited herein. Furthermore, all references, including patent applications, cited herein are hereby incorporated by reference to the same extent as if each reference were individually and specifically indicated to be incorporated by reference and were set forth in its entirety herein.

According to the present invention as disclosed herein, method of treating a patient in need of cabazitaxel comprising:
parenterally administering to said patient an aqueous composition of cabazitaxel comprising, cabazitaxel or a pharmaceutically acceptable salt thereof and treatment is substantially free of premedication, required to reduce the hypersensitivity reactions associated with excipients like surfactants.

As described in literature, Jevtana kit administration includes a premedication therapy before 30 minutes of administration of Cabazitaxel, the premedication includes an antihistamine dexchlorpheniramine or diphenhydramine and the like, a corticosteroid such as dexamethasone and the like and an H2 antagonist such as ranitidine.

Premedication therapy is an important prerequisite to reduce the hypersensitivity reactions associated with the surfactants.

Matthieu Picard et al., Clinic Rev Allerg Immunol, April 2014 titled Re-visiting Hypersensitivity Reactions to Taxanes: A Comprehensive Review discloses, although less common than immediate HSRs, a range of delayed, possibly immune-mediated, reactions have been described with taxanes. Importantly, delayed onset skin eruptions (most commonly a benign maculopapular rash) can be the prelude to an immediate HSR during the next infusion.

### Mechanisms of Immediate Hypersensitivity Reactions to Taxanes:

Three different mechanisms may account for immediate HSRs to taxanes:
(1) complement activation induced by Cremophor EL (compounded with paclitaxel) and polysorbate 80 (compounded with docetaxel) which generates anaphylatoxins triggering basophil/mast cell activation;
(2) histamine release through a direct but undefined effect of paclitaxel on basophils;
(3) an IgE/IgG-mediated mechanism directed against either the taxane moiety (paclitaxel, docetaxel) or the solvent (Cremophor EL, polysorbate 80).

The inventors have thoroughly investigated the need for a therapy, that is free of premedication to reduce the intake of the medications during or prior to administration of Cabazitaxel treatment to the patient and in turn increasing the compliance to a patient.

In an embodiment, the cabazitaxel treatment is substantially free of premedication to reduce the intake of the medications during or prior to administration of Cabazitaxel treatment to the patient and in turn increasing the compliance to the patient.

In an embodiment, the cabazitaxel treatment is substantially free of premedication to reduce the intake of the medications prior to administration of Cabazitaxel treatment to the patient and in turn increasing the compliance to the patient.

Cabazitaxel dosage administration includes the premedication leading inconvenience and cumbersome to administer large number of medications to the patients, which are not economical, hence the inventors have found a treatment therapy that is convenient, economical and less cumbersome to the patient.

In an embodiment, the present inventors have found an alternate and improved therapy to premedication therapy that is free of antihistamine, prior to the administration of Cabazitaxel composition.

In an embodiment, the present inventors have found a premedication therapy that is free of antihistamine, prior to the administration of cabazitaxel composition, the antihistamine includes dexchlorpheniramine, diphenhydramine, prochlorperazine and the like or combinations thereof.

In an embodiment, cabazitaxel treatment is optionally free of premedication therapy comprising of corticosteroids prior to the administration of cabazitaxel composition.

In an embodiment, cabazitaxel treatment is optionally free of premedication therapy comprising of corticosteroids such as dexamethasone prior to the administration of cabazitaxel composition.

In an embodiment, cabazitaxel treatment is optionally free of premedication therapy such as a corticosteroid, prior to the administration of cabazitaxel composition.

In an embodiment, cabazitaxel treatment is optionally free of premedication therapy such as a dexamethasone and the like, prior to the administration of cabazitaxel composition.

In an embodiment, cabazitaxel treatment comprises administration of premedication therapy such as an H2 antagonist and the like, prior to the administration of cabazitaxel composition.

In an embodiment, cabazitaxel treatment comprises administration of premedication therapy with ranitidine and the like, prior to the administration of cabazitaxel composition.

In a further embodiment the premedication therapy is administered to reduce the hypersensitivity reactions associated with excipients like surfactants.

According to another embodiment, the composition is free of surfactants to reduce the hypersensitivity reactions associated with surfactants, wherein surfactants are selected but not limited to polyethoxylated sorbitan fatty ester such as polyoxyethylene sorbitan monooleate (Polysorbate 80), its derivatives or mixtures thereof, polyethoxylated castor oils (Cremophor) its derivatives or mixtures thereof. In one embodiment, the method of treatment as disclosed herein, comprises parenteral administration of an aqueous composition of cabazitaxel that is free of surfactants and/or cremophor.

In one embodiment of the present invention, the aqueous composition of cabazitaxel is free of surfactants wherein the surfactants are selected from a group consisting of but not limited to polyethoxylated sorbitan fatty ester such as polyoxyethylene sorbitan monooleate (polysorbate 80), its derivatives or mixtures thereof, free of polyethoxylated castor oils its derivatives or mixtures thereof.

In an embodiment the invention includes method of treating a patient in need of cabazitaxel treatment, substantially free of premedication to control complement activation, reduce the toxicity and hypersensitivity reactions associated with the surfactants and/or cremophor.

In a further embodiment, the present invention does not cause complement activation leading to generation of anaphylatoxins and HSR's associated with excipients like surfactants and/or cremophor.

In another embodiment, the invention relates to a method for improving the cabazitaxel treatment to a patient in need of cabazitaxel comprising, parenterally administering to said patient an aqueous composition of cabazitaxel comprising, cabazitaxel or a pharmaceutically acceptable salt thereof and treatment is substantially free of premedication, required to reduce the hypersensitivity reactions associated with excipients like surfactants.

In an embodiment, the cabazitaxel treatment is substantially free of premedication to reduce the intake of the medications during or prior to administration of Cabazitaxel treatment to the patient and in turn increasing the compliance to the patient.

In an embodiment, the present inventors have found a premedication therapy that is free of antihistamine, prior to the administration of cabazitaxel composition, antihistamine includes dexchlorpheniramine, diphenhydramine, prochlorperazine and the like or combinations thereof.

In an embodiment, the present inventors have found an alternate therapy to premedication therapy that is free of antihistamine, prior to the administration of cabazitaxel composition.

In an embodiment, the present inventors have found a premedication therapy that is substantially free of antihistamine, prior to the administration of Cabazitaxel composition, antihistamine includes dexchlorpheniramine, diphenhydramine, prochlorperazine and the like or combinations thereof.

In an embodiment, the present inventors have found an alternate therapy to premedication therapy that is substantially free of antihistamine, prior to the administration of Cabazitaxel composition.

In an embodiment, cabazitaxel treatment is optionally free of premedication therapy such as a corticosteroid, prior to the administration of cabazitaxel composition.

In an embodiment, cabazitaxel treatment is optionally free of premedication therapy such as a dexamethasone and the like, prior to the administration of cabazitaxel composition.

In an embodiment, cabazitaxel treatment comprises of administration of premedication therapy such as an H2 antagonist and the like, prior to the administration of cabazitaxel composition.

In an embodiment, cabazitaxel treatment comprises administration of premedication therapy such as ranitidine and the like, prior to the administration of cabazitaxel composition.

In a further embodiment, the premedication therapy is administered to reduce the hypersensitivity reactions associated with excipients like surfactants.

In a further embodiment, the present invention does not cause complement activation leading to generation of anaphylatoxins and HSR's associated with excipients like surfactants and cremophors.

In a further embodiment, the present invention comprising the composition is free of surfactants does not cause complement activation leading to generation of anaphylatoxins and HSR's associated with excipients like surfactants and cremophors.

According to another embodiment, the composition is free of surfactants to reduce the hypersensitivity reactions associated with surfactants, wherein surfactants are selected but not limited to polyethoxylated sorbitan fatty ester such as polyoxyethylene sorbitan monooleate (polysorbate 80), its derivatives or mixtures thereof, polyethoxylated castor oils its derivatives or mixtures thereof.

In one embodiment, the method of treatment as disclosed herein, comprises parenteral administration of an aqueous composition of cabazitaxel that is free of surfactants and/or cremophor.

In one embodiment of the present invention, the aqueous composition of cabazitaxel is free of surfactants selected from a group consisting of but not limited to polyethoxylated sorbitan fatty ester such as polyoxyethylene sorbitan monooleate (polysorbate 80), its derivatives or mixtures thereof, polyethoxylated castor oils its derivatives or mixtures thereof.

In another embodiment the inventors have incorporated herein by reference WO2018109731 A1 disclosing the Cabazitaxel with cyclodextrin composition and have taken complete authorization to include this reference in this application.

In an alternative embodiment, the method of treatment as disclosed herein comprises parenteral administration of an aqueous composition comprising cabazitaxel and cyclodextrin derivative.

Cyclodextrin derivatives include but not limited to beta-cyclodextrin, gama-cyclodextrin, hydxoxyalkyl-beta-cyclodextrin, hydxoxypropyl-betacyclodextrin, hydroxypropyl-gama-cyclodextrin, dimethyl-beta-cyclodextrin, methylated-beta-cyclodextrin, carboxymethyl-beta-cyclodextrin, sulfoalkyl-betacyclodextrin, sulfobutyl-ether-beta-cyclodextrin (SBECD), modified cyclodextrins or its derivatives or any mixtures thereof.

Intravenously administered cyclodextrins (CDs) disappear rapidly from systemic circulation and are renally excreted intact. Systemically absorbed CDs distribute mainly in the extracellular compartments, and no deep compartments or storage pools are involved. Most CDs disappear from tissues dramatically within the first several hours of administration.

The safety of SBECD is very well established and is an enabling excipient in about 10 FDA-approved parenteral drug formulations with broad safety profile [Because of the high clearance and small Vd values, half-lives (t ½) of SBCD is quite short. Hence immediately after IV administration, it will be appearing in the proximal tubules of the kidney].

In one embodiment of the present invention, the aqueous composition comprises cabazitaxel or its pharmaceutically acceptable salt and sulfobutyl ether cyclodextrin (SBECD) in its formulation.

In another embodiment of the present invention, the aqueous composition comprises cabazitaxel or its pharmaceutically acceptable salt, a cyclodextrin derivative and optionally other pharmaceutically acceptable excipients wherein the excipients include acidifying agent, pH modifier, chelating agent, solvent, co-solvent, solubilizer, buffers, tonicity modifier, antioxidant, preservative and the like or any mixtures thereof.

Acidifying agents or pH modifier include but not limited to ascorbic acid, boric acid, citric acid, tartaric acid, lactic acid, oxalic acid, formic acid, benzene sulphonic acid, benzoic acid, maleic acid, malonic acid, glutamic acid, succinic acid, aspartic acid, diatrizoic acid, acetic acid and the like or mixtures thereof.

Chelating agent include but not limited to ethylene diamine tetraacetic acid (EDTA) and acceptable salts thereof, ethylene glycol-bis(beta-aminoethyl ether)-N,N,N′,N′-tetraacetic acid (EGTA) and acceptable salts thereof, and 8-Amino-2-[(2-amino-5-methylphenoxy) methyl]-6-methoxyquinoline-N,N,N′,N′-tetraacetic acid, tetrapotassium salt (QUIN-2) and acceptable salts thereof. Solvent or co-solvent include but not limited to alkanols, water; lower alkanols like C1-C4 alkanols; it includes ethanol, dehydrated alcohol, absolute alcohol and the like or any mixtures thereof.

Tonicity modifier include those commonly used in the parenteral preparations but not limited to sodium chloride, dextrose, mannitol, glycerol and the like or any mixtures thereof.

Antioxidant or preservative include but not limited to ascorbic acid, acetylcysteine, sulfurous acid salts (bisulfite, metabisulfite), monothioglyercol, phenol, metacresol, benzyl alcohol, parabens (methyl, propyl, butyl), benzalkonium chloride, chlorobutanol, thimerosal and the like or any mixtures thereof.

In one embodiment, the method of treatment comprises administration of an aqueous composition of cabazitaxel wherein the solvent is selected from water for injection, alkanol, water for injection; solubilizer include cyclodextrin and its derivatives; buffers include ascorbic acid, boric acid, citric acid, tartaric acid, lactic acid, oxalic acid, formic acid and the like, preservatives include ascorbic acid, acetylcysteine, sulfurous acid salts (bisulfite, metabisulfite), monothioglyercol, phenol, metacresol, benzyl alcohol, parabens (methyl, propyl, butyl), benzalkonium chloride, chlorobutanol, thimerosal and combinations thereof.

In one embodiment of the present invention, the method of treatment comprises parenteral administration of an aqueous composition of cabazitaxel or its pharmaceutically acceptable salt comprising a cyclodextrin derivative, sulfobutyl ether cyclodextrin (SBECD) and optionally other acceptable excipients wherein the excipients include a solvent selected from water for injection or alkanol; solubilizer include cyclodextrin and its derivatives; buffers include ascorbic acid, boric acid, citric acid, tartaric acid, lactic acid, oxalic acid, formic acid and the like, preservatives include ascorbic acid, acetylcysteine, sulfurous acid salts (bisulfite, metabisulfite), monothioglyercol, phenol, metacresol, benzyl alcohol, parabens (methyl, propyl, butyl), benzalkonium chloride, chlorobutanol, thimerosal and combinations thereof.

In another aspect, the method of treatment as disclosed herein, comprises parenteral administration of an aqueous composition of cabazitaxel wherein the composition is, liquid composition, ready to dilute, ready to use, ready to infuse compositions and the like.

In one embodiment of the present invention, the pharmaceutical composition is a ready to use and/or ready to infuse formulation. A ready to use composition is a formulation that is suitable for administration with or without further dilution.

In one embodiment, the pharmaceutical composition is a ready to dilute formulation. A ready to dilute composition is a formulation that is suitable for administration after dilution with a suitable diluent.

Suitable diluents include, but are not limited to, water, saline, dextrose, water for injection, bacteriostatic water for injection, and the like or combinations thereof.

The compositions have a pH from about 2 to about 8, example between about 2.5 to about 7.5.

The concentration of cabazitaxel used in the composition is between about 0.1 mg/mL to about 7.0 mg/ml.

The composition would reduce the handling difficulties and reconstitution challenges. Ready to use and/or ready to dilute compositions will minimize dosing errors resulting from reconstitution steps. Hence, ready to use and/or ready to dilute compositions reduce the time required for administration.

In one embodiment, ready to use and/or ready to dilute compositions may be in the concentration range from about 0.1 mg/mL to about 7 mg/mL and preferred volume of not more than about 700 mL.

In one embodiment of the present invention, the method of treatment comprises parenteral administration of aqueous composition of cabazitaxel or its pharmaceutically acceptable salt wherein the recommended dose is 20 mg/m² administered as a one-hour intravenous infusion every three weeks and a dose of 25 mg/m² used in select patients at the discretion of the treating healthcare provider.

The pharmaceutical composition according to the above embodiments is used to treat cabazitaxel sensitive diseases including but not limited to cancer, carcinoma or malignant disease such as carcinoma of the ovary, prostate cancer, breast cancer, and related conditions thereof.

In another embodiment the present invention is presented in an ampoule, a vial, IV bag, or a suitable parenteral container that provides the composition comprising Cabazitaxel or its pharmaceutical salt, cyclodextrins derivative, and optionally with other pharmaceutically acceptable excipients, the composition is free of surfactants.

The entire disclosure of each of the patent documents and scientific articles referred to herein is incorporated by reference for all purposes.

The invention may be embodied in other specific forms without departing from the spirit or essential characteristics thereof. The foregoing embodiments are therefore to be considered in all respects illustrative rather than limiting on the invention described herein.

### EXAMPLES

### Example 1: Cabazitaxel Composition [Ready to Dilute and/or Ready to Infuse Compositions]

| SI. No | Ingredients | Quantity mg/mL | |
|---|---|---|---|
| 1. | Cabazitaxel | 5 | mg |
| 2. | Dehydrated Alcohol | 0.04 | mg |
| 3. | SBECD | 600 | mg |
| 4. | Sodium metabisulfite | 3 | mg |

| | | |
|---|---|---|
| 5. | Water for injection | Qs to 1 mL |

### Manufacturing Procedure:

1. Phase-1: Collect batch quantity of ethanol, add and dissolve batch quantity of Cabazitaxel API.
2. Phase-2: Collect 60% batch size of nitrogen purged water for injection (WFI), add and dissolve batch quantity of SBECD (Sulfobutylether-β-cyclodextrin) under continuous stirring.
3. Add Phase-1 to Phase-2 under stirring at room temperature.
4. Add batch quantity of sodium meta bisulphite and dissolved under stirring.
5. Evaporate the ethanol by using nitrogen purging under continuous stirring.
6. Makeup the volume to 100% batch size using nitrogen purged Water for Injection (WFI) and stirr it for 30 to 45 minutes.
7. Filter the solution through 0.22p filter.
8. Fill the filtered solution into suitable containers, stopper, sealed and label the vials.

### Stability Details of Composition:

| | Time Points | | |
|---|---|---|---|
| | | 25° C./60% RH | |
| Test Parameters | Specification | (6 M) | 2-8° C. 6 M |
| Description | Report the value | *Complies | *Complies |
| pH | | 4.05 | 3.83 |

Related Substances

| | | | |
|---|---|---|---|
| 7,10-Dimethoxy | NMT 0.4% | 0.03 | 0.02 |
| DAB | | | |
| Tiglyol | NMT 0.4% | 0.22 | 0.05 |
| Cabazitaxel | | | |
| TES Cabazitaxel | NMT 0.4% | ND | ND |
| Any Unspecified | NMT0.20% | 0.13 | 0.05 |
| Impurity | | | |
| Total Impurities | NMT 2.0% | 0.54 | 0.19 |

*Clear colorless slightly viscous solution

In view of the above stability details, it is evident that the proposed composition is stable, ready to use and administer for the patients unlike that of the available compositions. The stability data results of both real time (2-8° C.) and accelerated condition (25° C./60% RH) were found to be satisfactory.

1. A method of treating a patient in need of cabazitaxel comprising;
parenterally administering to said patient an aqueous composition of cabazitaxel comprising, cabazitaxel or a pharmaceutically acceptable salt thereof, wherein said method is substantially free of premedication therapy.

2. The method according to claim 1, wherein premedication therapy is free of antihistamine, prior to the administration of cabazitaxel compositions.

3. The method according to claim 2, wherein antihistamine includes dexchlorpheniramine, diphenhydramine, prochlorperazine.

4. The method according to claim 1, wherein the premedication therapy is administered to reduce the hypersensitivity reactions associated with excipients like surfactants.

5. The method according to claim 4, wherein the surfactants are selected from polysorbate 80, polyethoxylated castor oil and/or combinations thereof.

6. A method for improving cabazitaxel treatment to a patient in need of cabazitaxel, the method comprising,
parenterally administering to said patient an aqueous composition of cabazitaxel, wherein the composition comprises, cabazitaxel or a pharmaceutically acceptable salt thereof and wherein the method is substantially free of premedication therapy.

7. The method according to claim 6, wherein premedication therapy is free of antihistamine, prior to the administration of cabazitaxel composition.

8. The method according to claim 7, wherein antihistamine includes, dexchlorpheniramine, diphenhydramine, prochlorperazine.

9. The method according to claim 6, wherein the premedication therapy is administered to reduce the hypersensitivity reactions associated with excipients like surfactants.

10. The method according to claim 9, wherein the surfactants are selected from polysorbate 80, polyethoxylated castor oil and/or combinations thereof.

11. The method according to claim 1 for treatment of Cabazitaxel sensitive diseases.

12. The method according to claim 1, wherein the aqueous composition further comprises excipients selected from solvents and/or cosolvents, solubilizer, pH modifiers, buffers, preservatives and combinations thereof.

13. The method according to claim 12, wherein the solvent is selected from water for injection, alkanol, water for injection; solubilizer include cyclodextrin and its derivatives; buffers include ascorbic acid, boric acid, citric acid, tartaric acid, lactic acid, oxalic acid, formic acid and the like, preservatives include ascorbic acid, acetylcysteine, sulfurous acid salts (bisulfite, metabisulfite), monothioglyercol, phenol, metacresol, benzyl alcohol, parabens (methyl, propyl, butyl), benzalkonium chloride, chlorobutanol, thimerosal and combinations thereof.

14. The method according to claim 6 for treatment of Cabazitaxel sensitive diseases.

15. The method according to claim 6, wherein the aqueous composition further comprises excipients selected from solvents and/or cosolvents, solubilizer, pH modifiers, buffers, preservatives and combinations thereof.

16. The method according to claim 15, wherein the solvent is selected from water for injection, alkanol, water for injection; solubilizer include cyclodextrin and its derivatives; buffers include ascorbic acid, boric acid, citric acid, tartaric acid, lactic acid, oxalic acid, formic acid and the like, preservatives include ascorbic acid, acetylcysteine, sulfurous acid salts (bisulfite, metabisulfite), monothioglyercol, phenol, metacresol, benzyl alcohol, parabens (methyl, propyl, butyl), benzalkonium chloride, chlorobutanol, thimerosal and combinations thereof.
